(19) United States
(12) Patent Application Publication
(10) Pub. No.: US 2016/0230141 A1
(43) Pub. Date: Aug. 11, 2016

(54) PROCESS FOR INCREASED YEAST BIOMASS

(71) Applicant: RESEARCH OF THE CITY UNIVERSITY OF NEW YORK, New York, NY (US)

(72) Inventors: Peter Lipke, Brooklyn, NY (US); Uthama Edupaganti, Jersey City, NJ (US)

(73) Assignee: RESEARCH FOUNDATION OF THE CITY UNIVERSITY OF NEW YORK, New York, NY (US)

(21) Appl. No.: 15/023,047

(22) PCT Filed: Sep. 18, 2014
(86) PCT No.: PCT/US2014/056276

§ 371 Date: Mar. 18, 2016

Related U.S. Application Data
(60) Provisional application No. 61/879,761, filed on Sep. 19, 2013.

(51) Int. Cl. C12N 1/38; C12N 1/32; C12N 1/16

(52) U.S. Cl. CPC C12N 1/38; C12N 1/16; C12N 1/32

(57) ABSTRACT

The present invention relates to a process for enhancing the growth and increasing the biomass of yeast cultures. The addition of ethanol during log growth phase increases the yield of yeast biomass and products purified from the biomass.

### BACKGROUND

The present invention relates to the field of biotechnology and yeast biomass. Yeast biomass has many applications. In the food industry it is seen as an excellent source of protein, nucleic acids and vitamins and useful to make bread, wine and beer. In non-food industries, such as the biofuel industry, it is used to produce ethanol. It has also been developed for human and veterinary medicine for the production of antibiotics, useful proteins and β-glucans. β-glucan is a biological immunomodulator such that it has the ability to prime and activate the immune system. Increasing the yield of yeast biomass has always been a challenge, therefore new methods and procedures are being developed to do so.

### SUMMARY OF THE INVENTION

The present invention is a process for increasing yeast biomass comprising culturing yeast cells in a growth medium comprising at least one fermentable carbon source and a non-fermentable carbon source.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows growth curves of S. cerevisiae in fermentor.

FIG. 2 shows a graph of the growth of S. cerevisiae at various media concentrations.

FIG. 3 shows a graph of the growth of S. cerevisiae using various carbon sources+ethanol.

FIGS. 4A, 4B and 4C show graphs of ethanol enhanced growth of various yeast strains.

FIG. 5 shows a graph of ethanol-enhanced growth curves using various media.

FIG. 6 shows a graph of glucan yield in yeast biomass.

### DETAILED DESCRIPTION OF ILLUSTRATIVE EMBODIMENTS

Efficient growth of yeast biomass requires the coordination of nutrient assimilation, energy generation, biosynthesis and cell division. Once nutrients are depleted to the point of reducing biosynthetic activity, yeast respond by decreasing their growth rate. Along these lines, a limitation to increasing yeast biomass involves carbon source preference. Carbon sources such as glucose and sucrose are metabolized rapidly as the preferred source of energy, and yeast enter stationary phase soon after utilization of these sugars, which limits biomass yield and the amount of yeast-derived natural products. This cessation of growth has been attributed to a metabolic stress that results from nutrient depletion and/or oxidative stress. The use of fed-batch methods only partially alleviates the problem. (Rocío Gómez-Pastor, Roberto Pérez-Torrado, Elena Garre and Emilia Matallana (2011). Recent Advances in Yeast Biomass Production, Biomass-Detection, Production and Usage, Dr. Darko Matovic (Ed.), ISBN: 978-953-307-492-4, InTech)

The current invention addresses this problem with the addition of ethanol, a non-fermentable carbon source, during early log phase growth. Addition of ethanol to yeast cultures facilitates better growth of any yeast strain grown on one or more carbon sources. In the presence of added ethanol, yeast continue to grow and metabolic consequences of carbon limitation appear to be absent. This result indicates that the yeast grow stress-free while simultaneously utilizing both fermentable and non-fermentable carbon sources in the media. Experiments were performed showing that in the presence of ethanol, yeast grow without apparent consumption of the non-metabolizable sugar. However, when a starter culture was grown in media containing fermentable carbon sources and switched to media containing ethanol as the only carbon source there was subsequent culture growth. Growth did not occur if the starter culture was not conditioned in media containing fermentable carbon sources.

In general, increasing the yield of yeast biomass involves adding ethanol to an exponentially growing culture of yeast, such as Saccharomyces cerevisiae, in early log phase under carbon-limited growth conditions. Seemingly, the ethanol is utilized as a non-fermentable carbon source in addition to the fermentable carbon source (glucose, sucrose, etc.) existing in the media. The fermentable carbon source generates ethanol throughout the growth period adding a metabolic and cellular stress. The addition of ethanol in early log phase results in up-regulation of anabolic pathways that utilize this non-fermentable carbon source, and thereby reduces the stress from the resulting products of fermentation. Thus, the yeast cells become competent to metabolize the non-fermentable and fermentable carbon sources simultaneously leading to increased biomass production.

### Example 1

A protocol for Cane Molasses Base Media (CMB) preparation was adapted from Demirci A et. al, (J Agric Food Chem. 1999 June; 47(6):2496-500). A liter of CMB was prepared based on the following composition:

| | | |
|---|---|---|
| CMF* | 7.5 | mL per liter |
| Na₂SO₄ | 1.13 | g per liter |
| CaCl₂•2H₂O | 0.14 | g per liter |
| MgCl₂•6H₂O | 0.94 | g per liter |
| KH₂PO₄ | 3.0 | g per liter |
| D-biotin | 0.4 | mg per liter |
| Trace element solution | 1 | mL per liter |

The Trace element solution was prepared as follows:

| | | |
|---|---|---|
| MgSO₄•7H₂O | 3.0 | g per liter |
| MnSO₄•H₂O | 0.5 | g per liter |
| NaCl | 1.0 | g per liter |
| FeSO₄•7H₂O | 0.1 | g per liter |
| CoSO₄•5H₂O | 0.18 | g per liter |
| CaCl₂•2H₂O | 0.08 | g per liter |
| ZnSO₄•7H₂O | 0.1 | g per liter |
| CuSO₄•5H₂O | 0.01 | g per liter |
| Al₂(SO₄)₃•nH₂O | 0.01 | g per liter |
| H₃BO₃ | 0.01 | g per liter |
| Na₂MoO₄•2H₂O | 0.01 | g per liter |

The solution was filter sterilized after preparation.

### *Cane Molasses Feeding Media (CMF):

| | | |
|---|---|---|
| Cane Molasses | 530 | g per liter |
| Urea | 21.7 | g per liter |
| CaCl₂•2H₂O | 0.14 | g per liter |
| NH₄H₂PO₄ | 4.0 | g per liter |
| MgCl₂•6H₂O | 0.94 | g per liter |
| KH₂PO₄ | 3 | g per liter |
| Trace Element solution | 1 | mL per liter |

*Urea & KH₂PO₄ filter sterilized & added to media after heat sterilization

In the following experiments, the concentration of CMF added to CMB media ranged from 2-4 times (1×-4×) the original, published concentration.

### Example 2

To grow in a shake flask, a starter culture was set up in a 250 ml flask with 50 ml of media. The culture was grown overnight at 30° C. and 170 rpm in a shaker incubator. The experimental cultures were inoculated by diluting the starter culture 1:5 or 1:10 to get a starting OD600 of 0.3 or 0.150, respectively. The cultures continued incubation at 30° C. and 170 rpm in a shaker incubator. When the OD600 of the culture reached 0.45 to 0.8, which is usually around two generations from inoculation, ethanol was added to a concentration of 2%. The culture was grown and additional OD's were taken periodically for a maximum of 120 hours.

### Example 3

For batch fermentation, a Sartorius BIOSTAT® Aplus bench top reactor equipped with Airflow gassing system, efficient agitation system, pH control and temp control was employed. The 1 L working volume vessel was equipped with air, alkali, acid and medium inlets ports. A temperature of 30° C., aeration of 1.3 l/min and agitation of 400 rpm was maintained throughout the experiment. The samples were taken and analyzed for cell density to establish growth curve of a Saccharomyces cerevisiae strain. Samples of the cultures were taken at 12, 24 and 32 hours to analyze the total biomass yield and glucan contents.

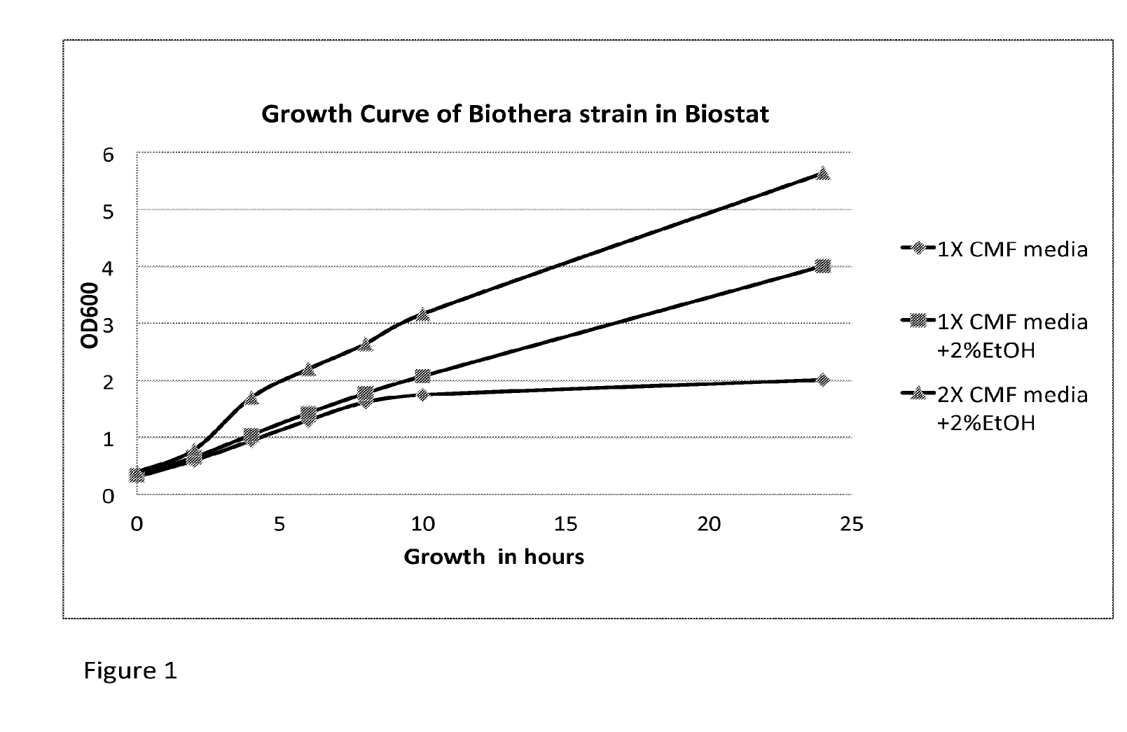

As shown in FIG. 1, S. cerevisiae grown in a fermentor in CMB-CMF medium supplemented with 2% ethanol shows increased growth rate and prolonged growth. In particular, 1×-CMB-CMF+2% ethanol media shows improved growth over media alone (control), while 2×-CMB-CMF+2% ethanol media provides even better growth rates and prolonged growth over the control.

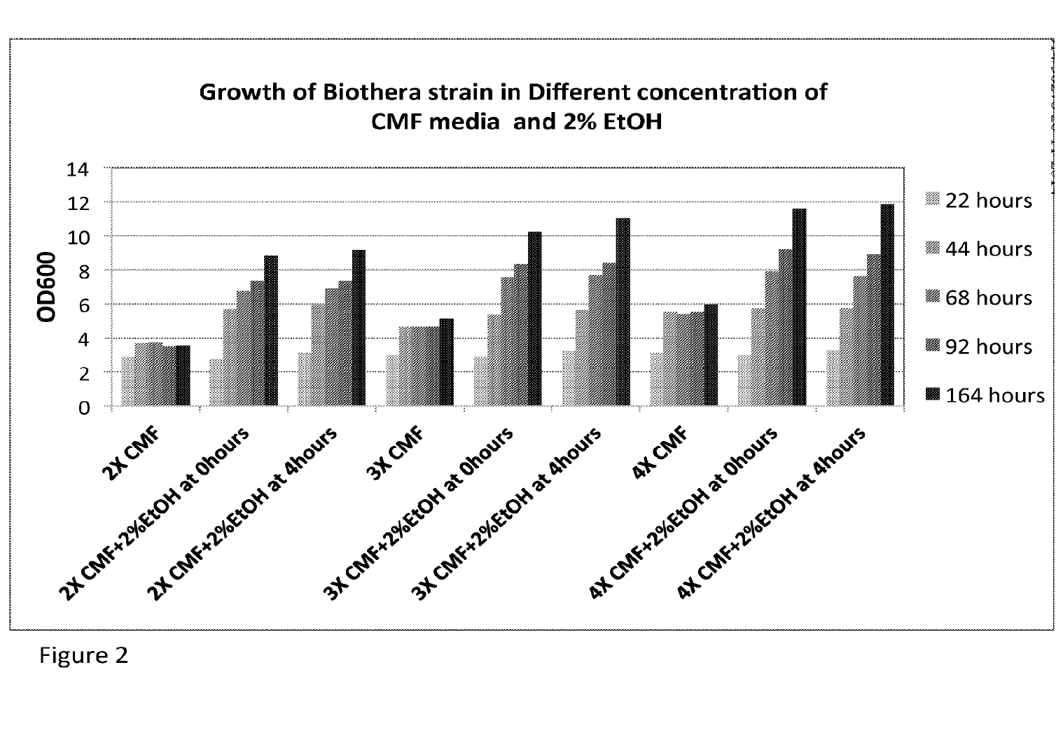

One way to optimize growth conditions and biomass volume is by varying the concentration of fermentable carbon sources. FIG. 2 shows the biomass results for a strain of S. cerevisiae grown in 2×, 3× and 4×CMF in CMF-CMB media with ethanol addition at either 0 hours and 4 hours of culture time.

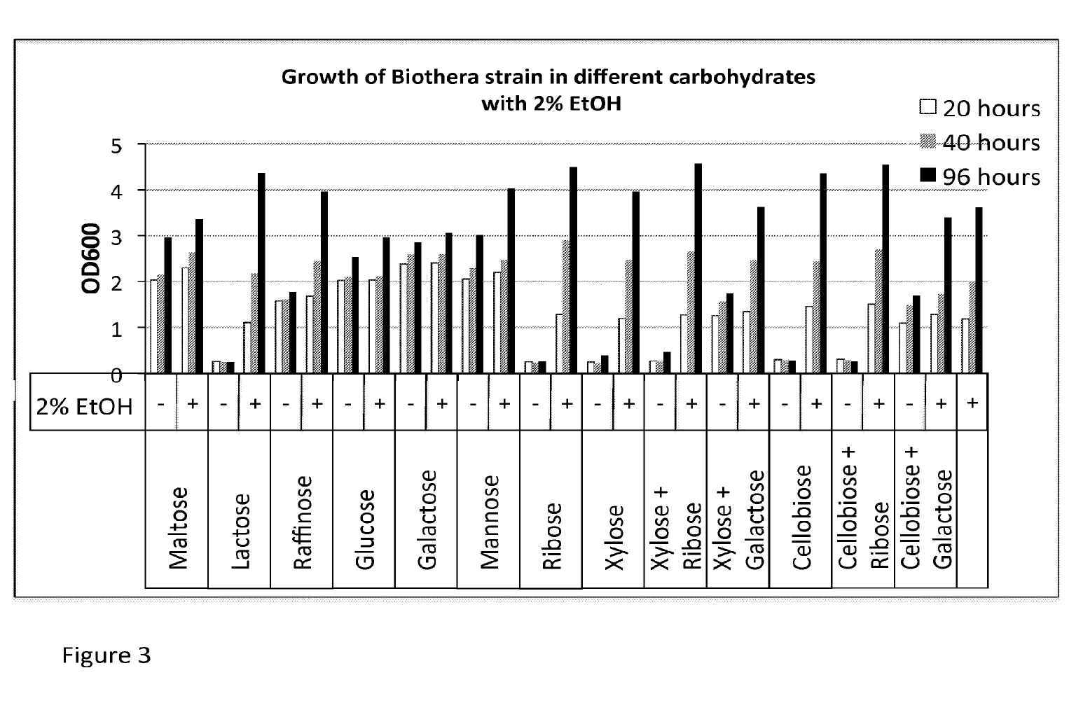

The benefit of adding ethanol to growing yeast cultures was shown for a number of different carbon sources. In FIG. 3, S. cerevisiae was grown in media containing maltose, lactose, raffinose, glucose, galactose, mannose, ribose, xylose, cellobiose and combinations thereof as carbon sources and either without (−) or with (+) ethanol. The results indicate that the addition of 2% ethanol to the cultures increased biomass production using any of the carbon sources. Thus, the benefit extends to any carbon source utilized in the media.

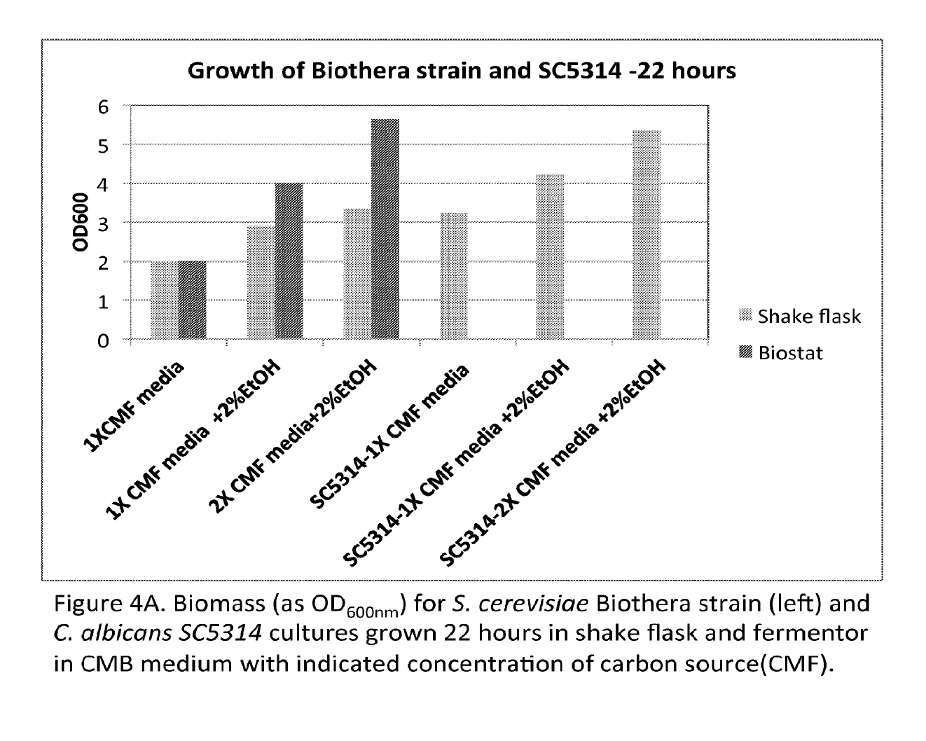

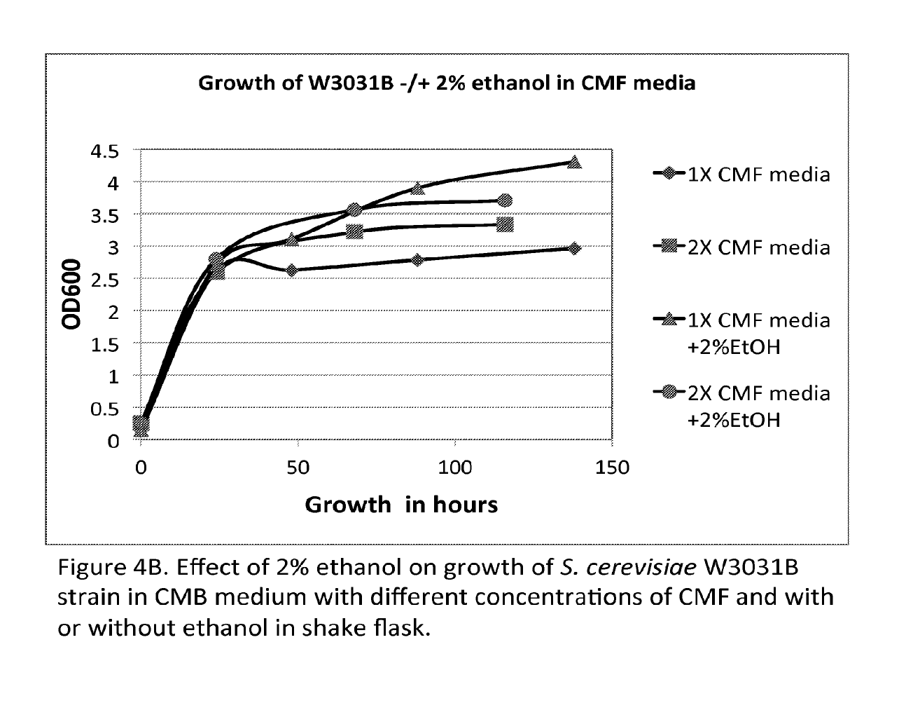

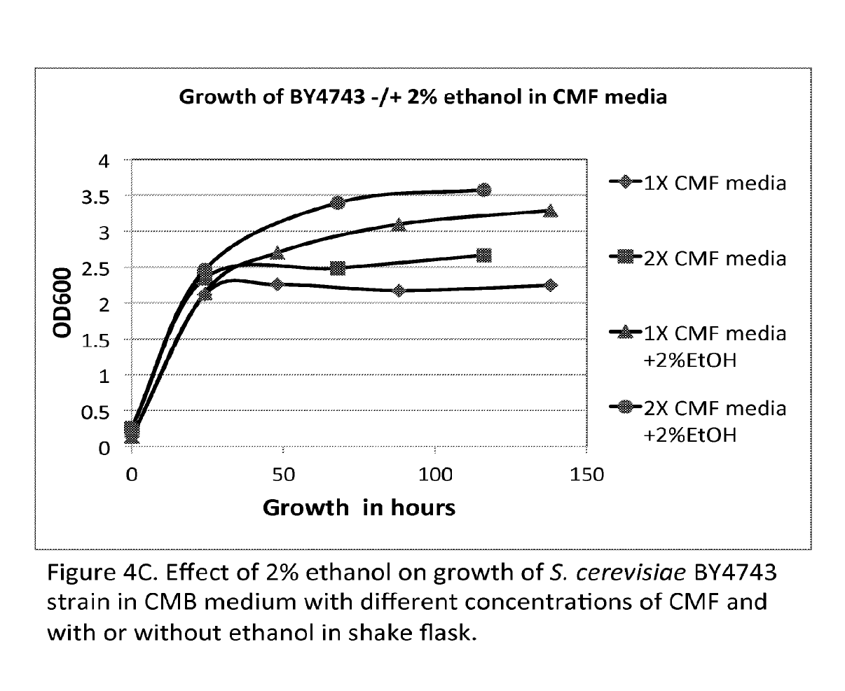

The benefit of ethanol addition to growth medium also applies to other yeast strains including, for example, S. cerevisiae, Candida albicans, BY4743 and W3031B. FIGS. 4A, 4B, and 4C show the results of enhanced biomass production of various yeast strains with the addition of 2% ethanol during growth. A proprietary S. cerevisiae strain, Candida albicans SC5314 and S. cerevisiae strains BY4743 and W303-1B were grown in CMF-CMB alone or CMF-CMB media supplemented with amino acids. The cultures of the proprietary strain and C. albicans SC5314 were grown for 22 hours, and S. cerevisiae BY4743 and W3031B were grown for 144 hours. FIG. 4A shows the growth of the proprietary strain at 22 hours in both a shake flask and a Biostat fermentor with or without 2% ethanol and 1× or 2×CMF media. It also shows the growth of SC5314−/+2% ethanol with 1× or 2×CMF media at 22 hours using the shake flask method. FIGS. 4B and 4C represent growth curves of S. cerevisiae W3031B and BY4743, respectively, using the shake flask method and measured from 0 to 144 hours, −/+2% ethanol with 1× or 2× CMF media. As is evident from the data, ethanol addition significantly enhanced yeast growth in cultures.

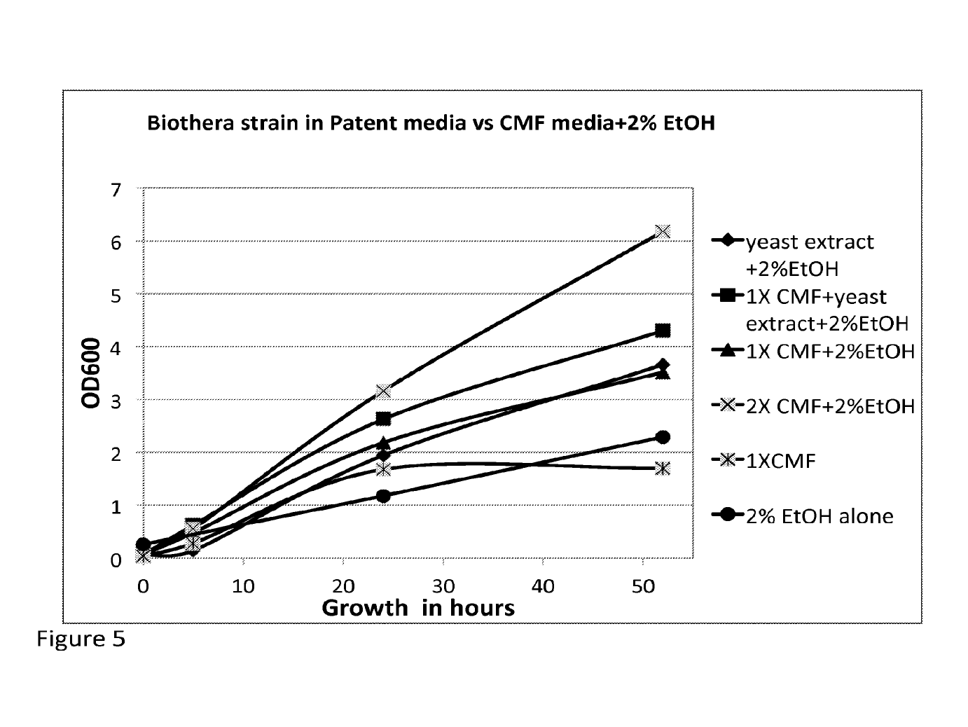

Ethanol-induced biomass yield and growth enhancement was also evident using any of a number of types of growth media. In FIG. 5, S. cerevisiae was grown in 2% ethanol and 2×CMF, 1×CMF, 1×CMF+yeast extract, yeast extract or yeast synthetic complete medium. A starter culture was grown in CMF-CMB media overnight. The starter inoculum was added to respective media as labeled in FIG. 5 to achieve a uniform starting OD. The cultures were incubated in a shaker incubator at 30° C. and 170 rpm for 4 to 5 hour or about 2 generations and then 2% ethanol was added. The cultures were incubated at 30° C. and OD₆₀₀ was measured at the time points shown in FIG. 5. Controls used were 1×CMF alone and 2% ethanol alone

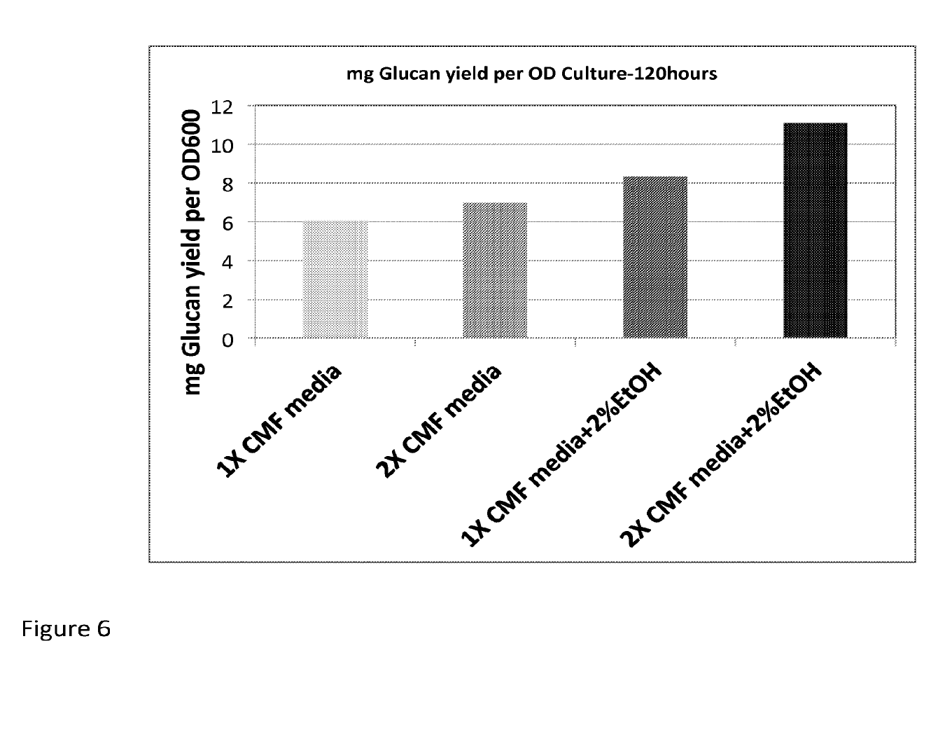

As shown in FIG. 6, growth in ethanol also increased the yield of β-glucan obtained from the biomass. The increased yield was due to increased biomass as well as increased yield of β-glucan per gram of biomass. A starter culture was grown in CMF-CMB media overnight. The starter inoculum was added to respective media as labeled in FIG. 6 to achieve a uniform starting OD. The cultures were incubated in a shaker incubator at 30° C. and 170 rpm for 4 to 5 hour or about 2 generations and then 2% ethanol was added. The cultures were incubated back at 30° C. and OD₆₀₀ was measured at 120 hours and the cultures were harvested. The culture pellets were alkali extracted for glucan and lyophilized. Dry weights of extracted glucans were measured. The glucan samples were subjected to GEM analysis for glucan content. Glucan yield per OD was calculated as (GEM glucan %*dry weight of extracted glucan)/(100*OD600 at 120 hours). For a standard S. cerevisiae strain, 2% ethanol gave the highest yields. However, ethanol concentrations between 0.5% and 4% are also beneficial under other conditions.

In summary, growth of yeasts in media containing a sugar, disaccharide, oligosaccharide or polysaccharide supplemented with ethanol increases growth rate, prolongs growth, increases biomass produced, and increases yield of specific products of yeast growth. This enhancement is seen in multiple yeast strains, species, and growth media. This finding promises to increase efficiency of industrial and research processes dependent on growing yeast, including, but not limited to, natural yeast products, yeast product foodstuffs, and biopharmaceuticals expressed by genetic modifications in yeast.

The complete disclosure of all patents, patent applications, and publications, and electronically available material cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

1. A process for increasing yeast biomass comprising culturing yeast cells in a growth medium comprising at least one fermentable carbon source and a non-fermentable carbon source.

2. The process of claim 1 wherein the non-fermentable carbon source is ethanol.

3. The process of claim 1 wherein the concentration of non-fermentable carbon source is between about 0.5% to 4%.

4. The process of claim 1 wherein the concentration of fermentable carbon source is between about 0.5% to 4%.

5. The process of claim 1 wherein the non-fermentable carbon source is added during lag, log or late exponential growth phase.

6. A process of increasing the growth rate and biomass of yeast strains engineered to metabolize naturally non-metabolized carbon sources comprising growing the yeast strains in media comprising a non-fermentable carbon source.

7. The process of claim 6 wherein the non-metabolized carbon source is one of pentoses, hexoses, disaccharides, trisaccharides, polysaccharides or combinations thereof.

8. The process according to claim 1 wherein the yeast are grown at a temperature other than from 24-37° C. in the presence of ethanol.

9. The process according to claim 1 further comprising tailoring the degree of agitation and aeration in the presence of ethanol to maximize the yield of the desired product.

10. The process of claim 2 wherein the concentration of non-fermentable carbon source is between about 0.5% to 4%.

11. The process according to claim 6 in that to grow yeast at different temperatures from 24-37° C. in the presence of ethanol to maximize the yield of the desired product.

12. The process according to claim 6 in that yeast can be grown with different degrees of agitation and aeration in the presence of ethanol to maximize the yield of the desired product.
